(19) United States
(12) Patent Application Publication
(10) Pub. No.: US 2020/0188006 A1
(43) Pub. Date: Jun. 18, 2020

(54) METHOD FOR CONTROLLING PRESSURE WITHIN INFLATABLE BALLOON OF INTRAVASCULAR CATHETER SYSTEM

(71) Applicant: Boston Scientific Scimed Inc, Maple Grove, MN (US)

(72) Inventor: Chadi Harmouche, Saint-Laurent (CA)

(21) Appl. No.: 16/796,081

(22) Filed: Feb. 20, 2020

Related U.S. Application Data
(63) Continuation of application No. PCT/US2018/039511, filed on Jun. 26, 2018.
(60) Provisional application No. 62/548,072, filed on Aug. 21, 2017.

(51) Int. Cl. A61B 18/02; A61M 25/10

(52) U.S. Cl. CPC A61B 18/02; A61B 2018/00577; A61M 25/1011; A61M 25/10186

(57) ABSTRACT

A method for controlling a balloon pressure of an inflatable balloon of an intravascular catheter system includes the steps of (i) sending sensor output to a controller, the sensor output being based at least partially on the balloon pressure, and (ii) maintaining the balloon pressure within a predetermined pressure range based at least partially upon the sensor output received by the controller. The step of maintaining includes one of (a) adjusting a flow rate of a cryogenic fluid through the inflatable balloon while moving the inflatable balloon from a first treatment site to a second treatment site, and (b) adjusting the flow rate of the cryogenic fluid that is selectively delivered from the fluid source to the inflatable balloon through an adjunct fluid injection line that is in fluid communication with a fluid exhaust line.

### CROSS-REFERENCE TO RELATED APPLICATION

This application is a continuation of International Application No. PCT/US2018039511, with an international filing date of Jun. 26, 2018, which claims priority to Provisional Application No. 62/548,072, filed Aug. 21, 2017 and titled “DEVICE AND METHOD FOR MAINTAINING PRESSURE WITHIN A CRYOBALLOON DURING THAWING”. As far as permitted, the contents of International Application No. PCT/US2018039511 and U.S. Provisional Application Ser. No. 62/548,072 are incorporated in their entirety herein by reference.

### TECHNICAL FIELD

The present disclosure relates to medical devices and methods for performing cryoablation procedures. More specifically, the disclosure relates to devices and methods for controlling pressure within the cryoballoon of a cryoablation catheter.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few.

In particular, catheter ablation involves delivering ablative energy to tissue inside the heart to block aberrant electrical activity from depolarizing heart muscle cells out of synchrony with the heart's normal conduction pattern. The catheter ablation procedure is performed by positioning a portion, such as a tip, of an energy delivery catheter adjacent to diseased or targeted tissue in the heart. The energy delivery component of the system is typically at or near the most distal (i.e., farthest from the operator or user) portion of the catheter, and often at the tip of the catheter.

Various forms of energy are used to ablate diseased heart tissue. These can include radio frequency, ultrasound and laser energy, to name a few. One form of energy that is used to ablate diseased heart tissue includes cryogenics (also referred to herein as “cryoablation”). During a cryoablation procedure, the tip of the catheter is positioned adjacent to targeted cardiac tissue, at which time energy is delivered in the form of a refrigerant or cryogenic fluid to create tissue necrosis, rendering the ablated tissue incapable of conducting electrical signals. The dose of energy delivered is a critical factor in increasing the likelihood that the treated tissue is permanently incapable of electrical conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis, while avoiding excessive energy leading to collateral tissue injury.

Atrial fibrillation, one of the most common arrhythmias, can be treated using catheter ablation. In the earliest stages of the disease, paroxysmal atrial fibrillation, the treatment strategy involves isolating the pulmonary vein(s) from the left atrial chamber of the heart. Recently, the use of techniques known as “balloon cryotherapy” catheter procedures to treat atrial fibrillation have increased. Some advantages of balloon cryotherapy include ease of use, shorter procedure times and improved patient outcomes. During balloon cryotherapy, an inflatable balloon at the distal end of the balloon catheter is positioned against the ostium of the pulmonary vein to occlude the pulmonary vein from blood flow. When balloon cryotherapy is used during a pulmonary vein isolation procedure, it is important that the inflatable balloon completely occludes blood flow from the pulmonary vein. In order to ensure effective positioning of the inflatable balloon, it generally takes several minutes and the use of guiding tools, such as a fluoroscopy or ICE (inter-cardiac echo).

Balloon cryotherapy can often include multiple ablations or ablation cycles on the same pulmonary vein or different pulmonary veins. When performing multiple ablations or ablation cycles on different pulmonary veins, fully deflating and inflating the inflatable balloon can cause a significant increase in the procedure time. Balloon cryotherapy procedures also generally include a thawing stage, which can be temperature based, time based, or both. During the balloon cryotherapy procedure, a cryogenic fluid, such as nitrous oxide, is injected into the inflatable balloon in order to freeze the diseased heart tissue. Once treated, the diseased heart tissue is allowed to thaw to a certain temperature and/or for a certain period of time. During the thawing stage, the inflatable balloon is maintained partially and/or fully inflated to reduce the likelihood of tissue damage to the patient and/or to reduce the need to reposition the balloon catheter. However, during the procedure it is not uncommon for minor leaks in the balloon catheter to form which can reduce the pressure within the inflatable balloon during thawing. Unfortunately, when a pressure within the inflatable balloon varies from a predetermined pressure value and/or outside of a predetermined pressure range, as a result of minor leaks or otherwise, the inflatable balloon can lose its positioning on the pulmonary vein. When this type of positioning loss occurs, the procedure time is not only increased due to the need to reposition the balloon catheter, but damage to the heart tissue and/or other surrounding tissue of the patient can occur.

### SUMMARY

The present invention is directed toward a method for controlling a balloon pressure of an inflatable balloon of an intravascular catheter system, the method including the steps of sending sensor output to a controller, the sensor output being based at least partially on the balloon pressure, and maintaining the balloon pressure within a predetermined pressure range based at least partially upon the sensor output received by the controller by adjusting a flow rate of a cryogenic fluid through the inflatable balloon while moving the inflatable balloon from a first treatment site to a second treatment site.

In some embodiments, the method can further include the step of positioning a pressure sensor within an inner balloon interior of the inflatable balloon. In other embodiments, the step of positioning can include positioning the pressure sensor within a fluid exhaust line.

In one embodiment, the step of maintaining can include adjusting the flow rate the cryogenic fluid moving through the fluid injection line. In another embodiment, the step of maintaining can include adjusting the flow rate of the cryogenic fluid moving through an adjunct fluid injection line that is in fluid communication with the fluid exhaust line. In still another embodiment, the step of maintaining can include adjusting the flow rate of the cryogenic fluid moving through the fluid exhaust line. In an alternative embodiment, the step of maintaining can include controlling with a control valve the flow rate of the cryogenic fluid moving through the fluid injection line. In another alternative embodiment, the step of maintaining can include controlling the cryogenic fluid moving through the adjunct fluid injection line that is in fluid communication with the fluid exhaust line. In still another alternative embodiment, the step of maintaining can include controlling the cryogenic fluid moving through the fluid exhaust line. The step of controlling can include at least partially opening the control valve with the controller based at least partially on the sensor output received by the controller. Alternatively, the step of controlling can include at least partially closing the control valve with the controller based at least partially on the sensor output received by the controller.

In certain embodiments, the method can further include the step of positioning the control valve on the fluid injection line. In other embodiments, the step of positioning can include the step of positioning the control valve on the adjunct fluid injection line. In yet other embodiments, the step of positioning can include the step of positioning the control valve on the fluid exhaust line.

In one embodiment, the method can also include the step of selectively delivering the cryogenic fluid from a fluid source to the inflatable balloon through the adjunct fluid injection line that is in fluid communication with the fluid exhaust line.

The present invention is also directed toward a method for controlling a balloon pressure of an inflatable balloon of an intravascular catheter system, the method including the steps of sending sensor output to a controller, the sensor output being based at least partially on the balloon pressure, and maintaining the balloon pressure within a predetermined pressure range based at least partially upon the sensor output received by the controller by adjusting a flow rate of a cryogenic fluid that is selectively delivered from a fluid source to the inflatable balloon through an adjunct fluid injection line that is in fluid communication with a fluid exhaust line.

In various embodiments, the method can include the step of delivering the cryogenic fluid to the inflatable balloon through a fluid injection line. Further, the method can also include the step of selectively removing the cryogenic fluid from the inflatable balloon through the fluid exhaust line.

In certain embodiments, the method can further include the step of positioning a pressure sensor within an inner balloon interior of the inflatable balloon. In other embodiments, the step of positioning can include positioning the pressure sensor within the adjunct fluid injection line.

In various embodiments, the step of maintaining can include controlling with a control valve the flow rate of the cryogenic fluid moving through the adjunct fluid injection line. Further, the step of controlling can include at least partially opening the control valve with the controller based at least partially upon the sensor output received by the controller. Alternatively, the step of controlling can include at least partially closing the control valve with the controller based at least partially upon the sensor output received by the controller.

In some embodiments, the method can further include the step of positioning the control valve on the adjunct fluid injection line.

Additionally, the present invention is further directed to a method for controlling a balloon pressure of an inflatable balloon of an intravascular catheter system, the method including the steps of sending sensor output to a controller, the sensor output being at least partially based on the balloon pressure, and maintaining the balloon pressure within a predetermined pressure range based at least partially upon the sensor output received by the controller by adjusting a flow rate of at least one of (i) a cryogenic fluid moving through a fluid injection line, (ii) the cryogenic fluid moving through an adjunct fluid injection line that is in fluid communication with a fluid exhaust line and (iii) the cryogenic fluid moving through the fluid exhaust line.

In certain embodiments, the step of maintaining can include controlling with a control valve the flow rate of at least one of (i) the cryogenic fluid moving through the fluid injection line, (ii) the cryogenic fluid moving through the adjunct fluid injection line and (iii) the cryogenic fluid moving through the fluid exhaust line.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic view of a patient and an embodiment of an intravascular catheter system having features of the present invention;

FIG. 2A is a simplified side view of a portion of the patient and one embodiment of a portion of the intravascular catheter system, including one embodiment of a balloon pressure maintenance assembly, positioned at a first treatment site;

FIG. 2B is a simplified side view of a portion of the patient and another embodiment of a portion of the intravascular catheter system, including another embodiment of the balloon pressure maintenance assembly, positioned at a second treatment site;

FIG. 3 is a simplified side view of a portion of the patient and still another embodiment of a portion of the intravascular catheter system including still another embodiment of the balloon pressure maintenance assembly;

FIG. 4 is a simplified side view of a portion of the patient and yet another embodiment of a portion of the intravascular catheter system including yet another embodiment of the balloon pressure maintenance assembly; and

FIG. 5 is a simplified side view of a portion of the patient and even another embodiment of a portion of the intravascular catheter system including even another embodiment of the balloon pressure maintenance assembly.

### DETAILED DESCRIPTION

Embodiments of the present invention are described herein in the context of a method for controlling a balloon pressure within an inflatable balloon of an intravascular catheter system. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound, pulsed DC electric fields and laser energy, as non-exclusive examples. The present invention is intended to be effective with any or all of these and other forms of energy.

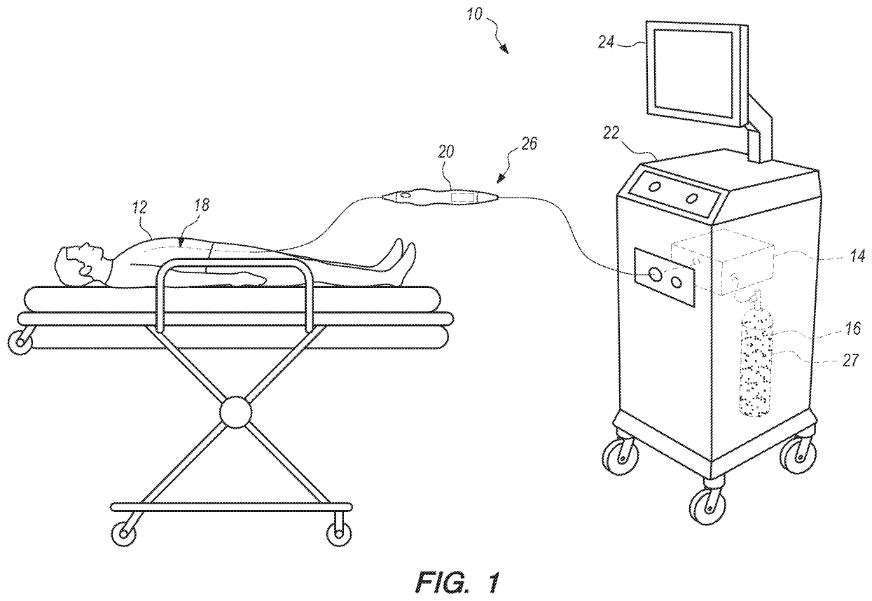

FIG. 1 is a schematic view of one embodiment of an intravascular catheter system 10 (also sometimes referred to as a “catheter system”) for use with a patient 12, which can be a human being or an animal. Although the catheter system 10 is specifically described herein with respect to the intravascular catheter system, it is understood and appreciated that other types of catheter systems and/or ablation systems can equally benefit by the teachings provided herein. For example, in certain non-exclusive alternative embodiments, the present invention can be equally applicable for use with any suitable types of ablation systems and/or any suitable types of catheter systems. Thus, the specific reference herein to use as part of the intravascular catheter system is not intended to be limiting in any manner.

The design of the catheter system 10 can be varied. In certain embodiments, such as the embodiment illustrated in FIG. 1, the catheter system 10 can include one or more of a control system 14, a fluid source 16 (e.g., one or more fluid containers), a balloon catheter 18, a handle assembly 20, a control console 22, a graphical display 24 (also sometimes referred to as a graphical user interface or “GUI”) and a balloon pressure maintenance assembly 26 (also sometimes referred to herein as a “pressure maintenance assembly”). It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional structures than those specifically illustrated and described herein.

In various embodiments, the control system 14 is configured to monitor and control the various processes of a cryoablation procedure. More specifically, the control system 14 can monitor and control release and/or retrieval of a cryogenic fluid 27 to and/or from the balloon catheter 18. The control system 14 can also control various structures that are responsible for maintaining or adjusting a flow rate and/or a pressure of the cryogenic fluid 27 that is released to the balloon catheter 18 during the cryoablation procedure. In such embodiments, the catheter system 10 delivers ablative energy in the form of cryogenic fluid 27 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive electrical signals, data and/or other information (also sometimes referred to as “sensor output”) from various structures within the catheter system 10. In various embodiments, the control system 14, the GUI 24 and/or the pressure maintenance assembly 26 can be electrically connected and/or coupled. In some embodiments, the control system 14 can receive, monitor, assimilate and/or integrate any sensor output and/or any other data or information received from any structure within the catheter system 10 in order to control the operation of the balloon catheter 18. Still further, or in the alternative, the control system 14 can control positioning of portions of the balloon catheter 18 within the body of the patient 12, and/or can control any other suitable functions of the balloon catheter 18.

The fluid source 16 (also sometimes referred to as “fluid container 16”) can include one or more fluid container(s) 16. It is understood that while one fluid container 16 is illustrated in FIG. 1, any suitable number of fluid containers 16 may be used. The fluid container(s) 16 can be of any suitable size, shape and/or design. The fluid container(s) 16 contains the cryogenic fluid 27, which is delivered to the balloon catheter 18 with or without input from the control system 14 during the cryoablation procedure. Once the cryoablation procedure has initiated, the cryogenic fluid 27 can be injected or delivered and the resulting gas, after a phase change, can be retrieved from the balloon catheter 18, and can either be vented or otherwise discarded as exhaust (not shown). More specifically, the cryogenic fluid 27 delivered to and/or removed from the balloon catheter 18 can include a flow rate that varies. Additionally, the type of cryogenic fluid 27 that is used during the cryoablation procedure can vary. In one non-exclusive embodiment, the cryogenic fluid 27 can include liquid nitrous oxide. In another non-exclusive embodiment, the cryogenic fluid 27 can include liquid nitrogen. However, any other suitable cryogenic fluid 27 can be used.

The design of the balloon catheter 18 can be varied to suit the design requirements of the catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a qualified healthcare professional (also referred to herein as an “operator”). As used herein, healthcare professional and/or operator can include a physician, a physician's assistant, a nurse and/or any other suitable person or individual. In certain embodiments, the balloon catheter 18 is positioned within the body of the patient 12 utilizing at least a portion of the sensor output that is received from the balloon catheter 18. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the operator to operate, position and control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid container 16 and the GUI 24. In some embodiments, the handle assembly 20 can integrate and/or include at least a portion of the control system 14 and/or pressure maintenance assembly 26 within an interior of the handle assembly 20. It is understood that the handle assembly 20 can include fewer or additional components than those specifically illustrated and described herein.

In the embodiment illustrated in FIG. 1, the control console 22 includes at least a portion of the control system 14, the fluid container 16 and/or the GUI 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the GUI 24.

In various embodiments, the GUI 24 is electrically connected to the control system 14. Additionally, the GUI 24 provides the operator of the catheter system 10 with information that can be used before, during and after the cryoablation procedure. For example, the GUI 24 can provide the operator with information based on the sensor output, and any other relevant information that can be used before, during and after the cryoablation procedure. The specifics of the GUI 24 can vary depending upon the design requirements of the catheter system 10, or the specific needs, specifications and/or desires of the operator.

In one embodiment, the GUI 24 can provide static visual data and/or information to the operator. In addition, or in the alternative, the GUI 24 can provide dynamic visual data and/or information to the operator, such as video data or any other data that changes over time, e.g., during the cryoablation procedure. Further, in various embodiments, the GUI 24 can include one or more colors, different sizes, varying brightness, etc., that may act as alerts to the operator. Additionally, or in the alternative, the GUI 24 can provide audio data or information to the operator.

As an overview, and as provided in greater detail herein, the pressure maintenance assembly 26 can be configured to maintain, measure and/or adjust the pressure of the cryogenic fluid 27 within the balloon catheter 18 during the cryoablation procedure. Additionally, the pressure maintenance assembly 26 can maintain or control the pressure of the cryogenic fluid 27 within the balloon catheter 18 during movement of the balloon catheter 18 between various locations within a circulatory system and/or heart of the patient 12.

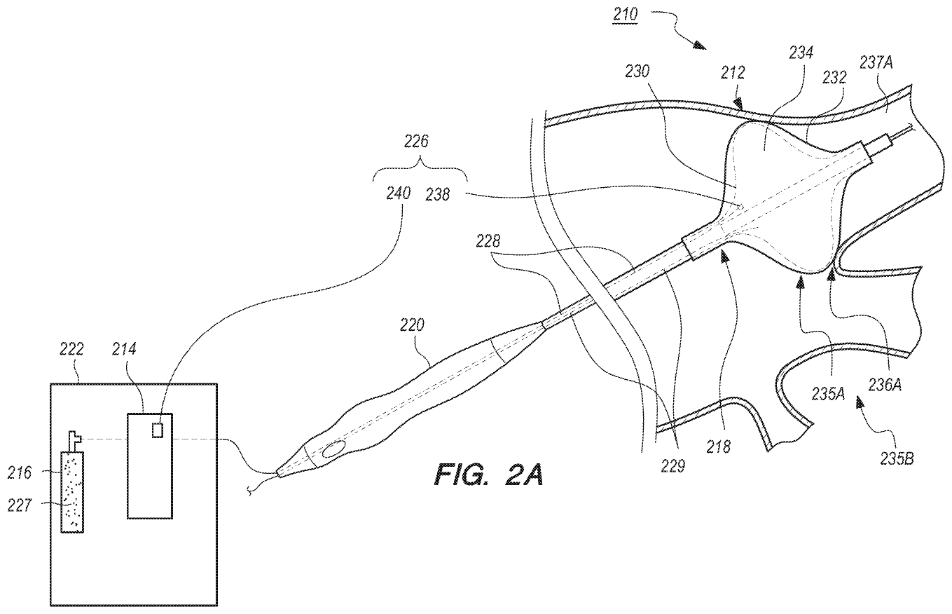

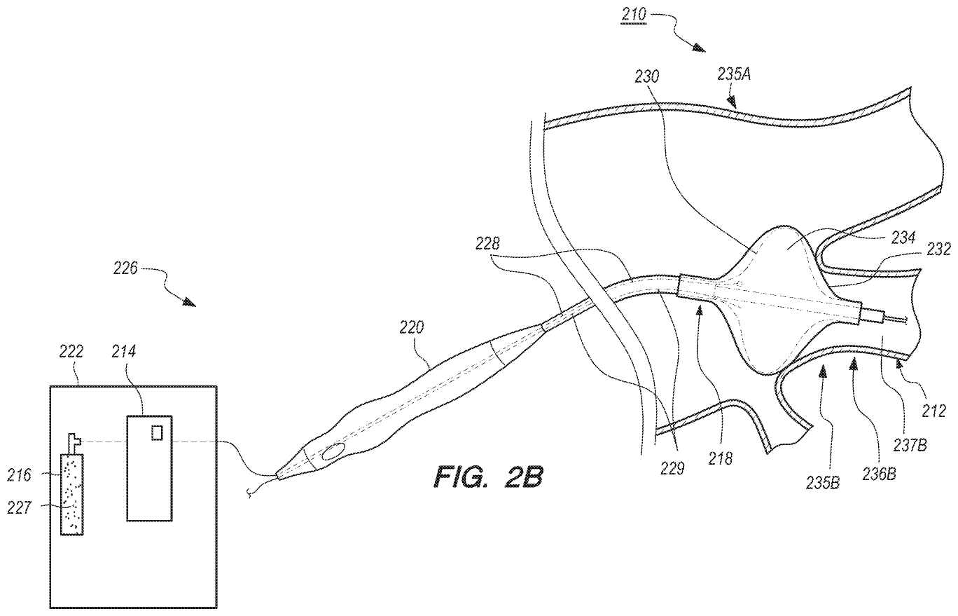

In the embodiment illustrated in FIG. 1, at least a portion of the pressure maintenance assembly 26 is integrated with the handle assembly 20. The portion of the pressure maintenance assembly 26 can be positioned at any suitable location within the handle assembly 20. Further, at least a portion of the pressure maintenance assembly 26 can be positioned within and/or outside of the handle assembly 20, such as within the balloon catheter 18 or control console 22, for example. Additionally, and/or in the alternative, at least a portion of the pressure maintenance assembly 26 can be included, positioned on, and/or integrated with any other suitable structure of the catheter system 10. The specific components and operations of the pressure maintenance assembly 26 will be described in greater detail herein below in relation to the embodiments illustrated in the FIGS. 2A-5. It is appreciated that the drawings included herewith may not necessarily be drawn to scale. Additionally, it is further appreciated that the drawings do not precisely represent the circulatory system and/or heart of the patient 12, but are included for purposes of clarity in demonstrating certain features and limitations of the catheter system 10.

FIG. 2A is a simplified side view of a portion of the patient 212 and one embodiment of a portion of the catheter system 210, including one embodiment of the pressure maintenance assembly 226. In the embodiment illustrated in FIG. 2A, the catheter system 210 can include the control system 214, the fluid source 216, the balloon catheter 218, the handle assembly 220, the control console 222, the pressure maintenance assembly 226, a fluid injection line 228 and a fluid exhaust line 229.

The fluid source 216 contains the cryogenic fluid 227 which is delivered to the balloon catheter 218 during the cryoablation procedure. In various embodiments, the cryogenic fluid 227 is delivered to the balloon catheter 218 via the fluid injection line 228. In some embodiments, once the cryoablation procedure has initiated, the cryogenic fluid 227 can be removed or retrieved from the balloon catheter 218, and can either be vented or otherwise discarded as exhaust via the fluid exhaust line 229.

The balloon catheter 218 is inserted into the body of the patient 212 during the cryoablation procedure. In this embodiment, the balloon catheter 218 includes an inner inflatable balloon 230 and an outer inflatable balloon 232 that substantially encircles and/or surrounds the inner inflatable balloon 230. The inner inflatable balloon 230 defines an inner balloon interior 234. It is recognized that the inner inflatable balloon 230 and the outer inflatable balloon 232 can also be referred to as a “first inflatable balloon” and a “second inflatable balloon”, and that either inflatable balloon 230, 232 can be the first inflatable balloon or the second inflatable balloon. It is also understood that the balloon catheter 218 can include other structures as well. However, for clarity these other structures have been omitted from FIG. 2A.

During the cryoablation procedure, the inner inflatable balloon 230 can be partially or fully inflated so that at least a portion of the inner inflatable balloon 230 expands toward and/or against a portion of the outer inflatable balloon 232 (although a space is shown between the inner inflatable balloon 230 and the outer inflatable balloon 232 in FIG. 2A for clarity). As provided herein, once the inner inflatable balloon 230 is sufficiently inflated, the outer inflatable balloon 232 can then be properly positioned within the patient 212 to abut and/or form a seal at a first treatment site 235A, i.e., the target tissue which includes relevant portion(s) of the circulatory system of the patient 212, such as a first ostium 236A of a first pulmonary vein 237A, as one non-exclusive example. While FIG. 2A, shows the balloon catheter 218 and/or the inflatable balloons 230, 232 positioned at the first treatment site 235A, it is understood that during the cryoablation procedure, the balloon catheter 218 and/or the inflatable balloons 230, 232 can be moved or positioned at a plurality of treatment sites 235A, 235B, i.e., a first treatment site 235A, a second treatment site 235B, a third treatment site, etc. In other words, a single cryoablation procedure can include treatment at various locations within the circulatory system of the patient 212.

In an alternative embodiment, the inner inflatable balloon 230 and the outer inflatable balloon 232 can be in a retracted position. This type of balloon assembly is sometimes referred to herein as a “tipless balloon assembly”. In one embodiment, the tipless balloon assembly can be made using the above described inner inflatable balloon 230 and outer inflatable balloon 232, assembled in tandem or individually to another portion of the balloon catheter 218. This structure provides a relatively compact shape, eliminating the approximately 8 to 13 mm tip from the total length of the tipless balloon assembly. Moreover, the reduction and/or elimination of the distal tip and/or the distal catheter end enables treatment at sites other than the pulmonary veins 237A, 237B where a distal tip would otherwise inhibit contact between the outer inflatable balloon 230 and cardiac tissue of the patient 12 (illustrated in FIG. 1). Thus, in this embodiment, the first treatment site 235A and/or the second treatment site 235B can be locations other than the pulmonary veins 237A, 237B, or can include one or more of the pulmonary veins 237A, 237B.

Additionally, as referred to herein, each cryoablation procedure can include various stages, which may include: (i) an inflation stage, (ii) an ablation stage, and (iii) a thawing stage, as non-exclusive examples. As utilized herein, “inflation stage” refers generally to the stage of the cryoablation procedure prior to the ablation stage, wherein the cryogenic fluid 227 is delivered from the fluid container 216 to the inflatable balloons 230, 232 with a flow rate that does not cause tissue necrosis. During inflation of the inflatable balloons 230, 232, the operator may adjust or position the inflatable balloons 230, 232 within the body of the patient 212 to achieve positioning of the inflatable balloons 230, 232 adjacent to the first treatment site 235A of the patient 212. As set forth herein, the first treatment site 235A can include at least a portion of the heart tissue of the patient 212 that is to be treated by the catheter system 210, such as the first ostium 236A and first pulmonary vein 237A, for example. Once positioned adjacent to the first treatment site 235A and the first pulmonary vein 237A is occluded, ablation of at the first treatment site 235A may be initiated.

The “ablation stage” refers generally to the stage of the cryoablation procedure when the cryogenic fluid 227 is delivered from the fluid container 216 to the inflatable balloons 230, 232 with the cryogenic fluid 227 having the flow rate to create tissue necrosis. Tissue necrosis has the effect of rendering targeted tissue incapable of conducting cardiac electrical signals. During ablation of the targeted tissue, the inflatable balloons 230, 232 are positioned adjacent to targeted tissue of the first treatment site 235A, with the first pulmonary vein 237A being occluded.

The “thawing stage” refers generally to the stage of the cryoablation procedure after the ablation stage when ablated heart tissue is allowed to thaw. In some embodiments, the thawing stage includes the cryogenic fluid 227 being delivered from the fluid container 216 to the inflatable balloons 230, 232 with the flow rate sufficient to maintain the inflatable balloons 230, 232 partially or substantially inflated to reduce the likelihood of the balloon catheter 218, including the inflatable balloons 230, 232, from falling out of position and/or to reduce the likelihood of tissue damage to the patient 212. Thawing can be temperature based, time based, or both. Temperature based means that the ablated heart tissue is allowed to thaw to a certain temperature. Time based means the ablated heart tissue is allowed to thaw for a certain period of time. The temperature and period of time can vary depending on the patient 212 and/or any other cryoablation parameters. Additionally, it is understood that each cryoablation procedure may include the same stage or any combination of stages. Additionally, and/or alternatively, it is further understood that the cryoablation procedure may also include other stages not specifically mentioned herein.

In the embodiments described herein, the inflatable balloons 230, 232 are partially or continuously injected with the cryogenic fluid 227 during the thawing stage to maintain a particular level of inflation of the inflatable balloons 230, 232. Alternatively, the inner inflatable balloon 230 and/or the outer inflatable balloon 232 can also be partially deflated during the thawing stage to maintain the particular level of inflation of the inflatable balloons 230, 232.

The fluid injection line 228 functions as a conduit through which the cryogenic fluid 227 is delivered from the fluid source 216 to the inner inflatable balloon 230, i.e., the inner balloon interior 234, during the cryoablation procedure. It is understood that while the fluid injection line 228 is illustrated in FIG. 2A, any suitable number of additional fluid injection lines or combination of fluid injection lines may be used. In some embodiments, such as the embodiment illustrated in FIG. 2A, the fluid injection line 228 can also function as a conduit through which the inner balloon interior 234 is partially or continuously injected with the cryogenic fluid 227 during the thawing stage in order to maintain the inner inflatable balloon at least partially inflated. In other words, the fluid injection line 228 is in fluid communication with the inner balloon interior 234. The cryogenic fluid 227 moving through the fluid injection line 228 can include a flow rate that varies depending on each stage of the cryoablation procedure.

The design of the fluid injection line 228 can vary. In certain embodiments, the fluid injection line 228 can include a relatively small diameter tube through which the cryogenic fluid 227 moves through the catheter system 210. In FIG. 2A, the fluid injection line 228 is shown to extend from the fluid source 216 to the inner balloon interior 234. In alternative embodiments, the fluid injection line 228 can be connected to and/or extend through other structures and/or components of the catheter system 210.

In various embodiments, the fluid exhaust line 229 functions as a conduit through which cryogenic fluid 227 within the inner inflatable balloon 230, i.e., the inner balloon interior 234, can be retrieved or removed as exhaust from the balloon catheter 218. In other words, the fluid exhaust line 229 can also be in fluid communication with the inner balloon interior 234. In alternative embodiments, the fluid exhaust line 229 can function as a conduit through which the cryogenic fluid 227 is selectively delivered to the inner balloon interior 234 during the thawing stage or any other stage of the cryoablation procedure. The cryogenic fluid 227 moving through the fluid exhaust line 229 can also include a flow rate that varies depending on each stage of the cryoablation procedure. In various embodiments, the flow rate of the cryogenic fluid 227 moving through the fluid exhaust line 229 can be substantially similar to the flow rate of the cryogenic fluid 227 moving through the fluid injection line 228. As used herein, use of the term “substantially” is intended to allow for minor deviations in the flow rate.

The design of the fluid exhaust line 229 can vary. In certain embodiments, the fluid exhaust line 229 can include a relatively small diameter tube through which the cryogenic fluid 227 moves. In the embodiment illustrated in FIG. 2A, the fluid exhaust line 229 is shown to extend from a location outside the handle assembly 220 to the inner balloon interior 234. In some embodiments, the fluid exhaust line 229 can be connected to and/or extend through various structures and/or components of the catheter system 210. For example, in one embodiment, the fluid exhaust line 229 can extend from a vacuum pump (not shown) to the inner balloon interior 234. In another embodiment, the fluid exhaust line 229 can extend from a portion of the control console 222 to the inner balloon interior 234.

The pressure maintenance assembly 226 provided herein maintains, measures and/or adjusts a balloon pressure within the inner balloon interior 234 during the cryoablation procedure. Additionally, the pressure maintenance assembly 226 can maintain or control the balloon pressure within the inner balloon interior 234 during treatment at the plurality of treatment sites 235A, 235B, such as when moving from the first treatment site 235A to the second treatment site 235B, for example. In the embodiments described herein, “balloon pressure” includes a pressure within the inner balloon interior 234 at or substantially contemporaneously with the time the pressure in the inner balloon interior 234 is measured. The pressure maintenance assembly 226 can function to maintain the balloon pressure in order to reduce the likelihood of the balloon catheter 218 from falling out of position on the first pulmonary vein 237A of the patient 212 during the cryoablation procedure. The pressure maintenance assembly 226 can also function to maintain the balloon pressure during treatment at the plurality of treatment sites 235A, 235B in order to facilitate a reduction of the cryoablation procedure time. For example, when moving between the plurality of treatment sites 235A, 235B during the cryoablation procedure, i.e., from the first treatment site 235A to the second treatment site 235B, for example, the pressure maintenance system 226 can maintain the inflatable balloons 230, 232 at least partially and/or fully inflated, which can limit the time generally required during the inflation stage.

Furthermore, as referred to herein, the balloon pressure can include a predetermined balloon pressure value and/or a predetermined balloon pressure range. The predetermined balloon pressure value can include a preset or predetermined minimum balloon pressure to maintain the positioning of the balloon catheter 218 on or near the first pulmonary vein 237A of the patient 212 during any stage of the cryoablation procedure, such as the thawing stage, or to maintain the inflatable balloons 230, 232 at least partially and/or fully inflated during treatment at the plurality of treatment sites 235A, 235B. For example, in one embodiment, the predetermined balloon pressure value can include the balloon pressure of at least approximately 1 psig. In non-exclusive alternative embodiments, the predetermined balloon pressure value can include the balloon pressure of at least approximately 2 psig, 3 psig, 4 psig or 5 psig, as non-exclusive examples. In yet another embodiment, the predetermined balloon pressure value can include the balloon pressure of less than 1 psig or greater than 5 psig. Alternatively, the predetermined balloon pressure value can include any other suitable balloon pressure that can maintain proper positioning of the balloon catheter 218 on the first pulmonary vein 237A of the patient 212 during the cryoablation procedure, or to maintain the inflatable balloons 230, 232 at least partially and/or fully inflated.

Additionally, the predetermined balloon pressure range can include a preset or predetermined balloon pressure range sufficient to maintain the positioning of the balloon catheter 218 on or near the first pulmonary vein 237A of the patient 212 during any stage of the cryoablation procedure, such as the thawing stage, or to maintain the inflatable balloons 230, 232 at least partially and/or fully inflated during treatment at the plurality of treatment sites 235A, 235B. For example, the predetermined balloon pressure range can include the balloon pressure greater than approximately 1 psig and less than approximately 10 psig. Alternatively, the balloon pressure can include greater than 10 psig or less than 1 psig. Further, the predetermined balloon pressure range can include any other suitable balloon pressure range that sufficiently maintains proper positioning of the balloon catheter 218 on the first pulmonary vein 237A of the patient 212 during the cryoablation procedure, or to maintain the inflatable balloons 230, 232 at least partially and/or fully inflated.

The design of the pressure maintenance assembly 226 can vary to suit the design requirements of the catheter system 210. In the embodiment illustrated in FIG. 2A, the pressure maintenance assembly 226 includes a pressure sensor 238 and a controller 240. In one embodiment, the pressure maintenance assembly 226 can include a PID system to maintain and/or adjust the balloon pressure during the cryoablation procedure. It is understood that the pressure maintenance assembly 226 can include fewer or additional components than those specifically illustrated and described herein.

In various embodiments, the pressure sensor 238 can sense, measure and/or monitor the balloon pressure within the inner balloon interior 234 during the cryoablation procedure, including during treatment at the plurality of treatment sites 235A, 235B. The design of the pressure sensor 238 can be varied. In certain embodiments, the pressure sensor 238 can transmit or send electronic and/or other signals, e.g. sensor output, to the controller 240. In some embodiments, the pressure sensor 238 can send the sensor output, which can be in the form of electrical signals, to the controller 240 via a transmission line (not shown). Alternatively, the pressure sensor 238 can send the sensor output to the controller 240 via any suitable manner or method. In the embodiment illustrated in FIG. 2A, the pressure sensor 238 is positioned within the inner balloon interior 234. However, the pressure sensor 238 can be positioned at any other location away from the inner balloon interior 234, i.e., at any other location outside the inner balloon interior 234 and within the catheter system 210.

The controller 240 is configured to receive and process the electronic or other suitable signals, e.g., sensor output, from the pressure sensor 238. The controller 240 can also control or adjust the flow rate of the cryogenic fluid 227 during the cryoablation procedure, including during treatment at the plurality of treatment sites 235A, 235B, based at least partially on the sensor output that has been received and processed by the controller 240. By controlling or adjusting the flow rate of the cryogenic fluid 227, the balloon pressure within the inner balloon interior 234 can be increased and/or decreased during the cryoablation procedure. For example, based at least in part on the sensor output, the controller 240 can process and/or determine whether the balloon pressure has varied from the predetermined balloon pressure value and/or is outside the predetermined balloon pressure range. In such embodiments, when the controller 240 determines that the balloon pressure is below the predetermined balloon pressure value and/or the predetermined balloon pressure range, the controller 240 can increase the balloon pressure by increasing the flow rate of the cryogenic fluid 227 moving through the fluid injection line 228. Alternatively, the controller 240 can increase the balloon pressure by decreasing the flow rate of the cryogenic fluid 227 moving through the fluid exhaust line 229. In other embodiments, when the controller 240 determines that the balloon pressure is above the predetermined balloon pressure value and/or predetermined balloon pressure range, the controller 240 can decrease the balloon pressure by decreasing the flow rate of the cryogenic fluid 227 moving through the fluid injection line 228. Alternatively, the controller 240 can decrease the balloon pressure by increasing the flow rate of the cryogenic fluid 227 moving through the fluid exhaust line 229.

In certain embodiments, such as the embodiment in FIG. 2A, the controller 240 can include or be integrated with the control system 14 (illustrated in FIG. 1). Alternatively, the controller 240 can be included or integrated with any other suitable structure of the catheter system 210, such as the handle assembly 220, for example.

In the embodiment illustrated in FIG. 2A, the cryogenic fluid 227 is delivered to the inner balloon interior 234 via the fluid injection line 228 during the cryoablation procedure. In this embodiment, the flow rate of the cryogenic fluid 227 moving within the fluid injection line 228 can be controlled and/or adjusted by the controller 240. In other embodiments, the cryogenic fluid 227 can be selectively delivered to the inner balloon interior 234 via other lines or conduits within the catheter system 210. For example, in one embodiment, during the thawing stage, the cryogenic fluid 227 can be delivered to the inner balloon interior 234 via the fluid exhaust line 229. In such example, the controller 240 can control and/or adjust the flow rate of the cryogenic fluid 227 moving within the fluid exhaust line 229, which may include the cryogenic fluid 227 being delivered and/or removed from the inner balloon interior 234. Alternatively, the cryogenic fluid 227 can be selectively delivered to the inner balloon interior 234 via any other suitable manner or method. Additionally, and/or in the alternative, the controller 240 can control or adjust the flow rate of the cryogenic fluid 227 via any suitable manner or method.

FIG. 2B is a simplified side view of a portion of the patient 212 and another embodiment of a portion of the catheter system 210, including another embodiment of the pressure maintenance assembly 226. In the embodiment illustrated in FIG. 2B, the catheter system 210 can include the control system 214, the fluid source 216, the balloon catheter 218, the handle assembly 220, the control console 222, the pressure maintenance assembly 226, the fluid injection line 228 and the fluid exhaust line 229. In FIG. 2B, the catheter system 210 functions in substantially the same manner as described in FIG. 2A. However, in this embodiment, the portion of the catheter system 210, i.e., the balloon catheter 218, is positioned at the second treatment site 235B.

In the embodiment illustrated in FIG. 2B, the pressure maintenance assembly 226 can maintain or control the balloon pressure within the inner balloon interior 234 when the cryoablation procedure is to be performed at more than one treatment site 235A, 235B. More specifically, the pressure maintenance assembly 226 can maintain the inflatable balloons 230, 232 at least partially and/or fully inflated when moving from the first treatment site 235A to the second treatment site 235B, for example. The second treatment site 235B can include at least a portion of the heart tissue of the patient 212 that is to be treated by the catheter system 210, such as a second ostium 236B of a second pulmonary vein 237B.

In FIG. 2B, the pressure maintenance system 226 can maintain the inflatable balloons 230, 232 at least partially and/or fully inflated when moving from the first treatment site 235A to the second treatment site 235B, which can limit the time generally required during the inflation stage. Once positioned adjacent to the second treatment site 235B and the second pulmonary vein 237B is occluded, ablation of at the second treatment site 235B may be initiated. Accordingly, in various embodiments, the pressure maintenance system 226 can have the effect of reducing the cryoablation procedure time.

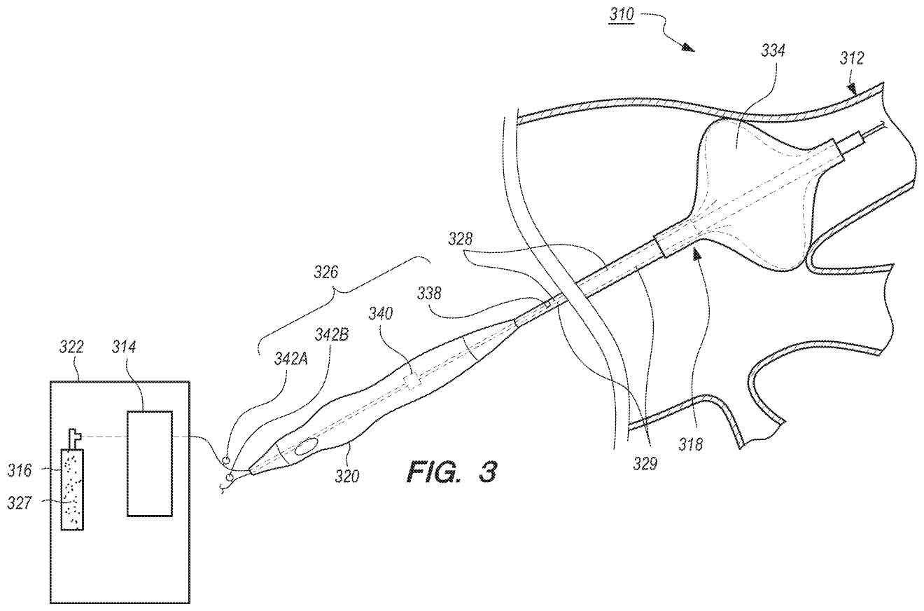

FIG. 3 is a simplified side view of a portion of the patient 312 and still another embodiment of a portion of the catheter system 310 including still another embodiment of the pressure maintenance assembly 326. In the embodiment illustrated in FIG. 3, the catheter system 310 includes the control system 314, the fluid source 316, the balloon catheter 318, the handle assembly 320, the control console 322, the pressure maintenance assembly 326, the fluid injection line 328 and the fluid exhaust line 329. However, in this embodiment, the pressure maintenance assembly 326 includes the pressure sensor 338, the controller 340 and one or more control valves 342A, 342B (i.e., first control valve 342A and second control valve 342B).

In certain embodiments, the control valves 342A, 342B can control and/or adjust the flow rate of the cryogenic fluid 327 moving through the fluid injection line 328 and/or the fluid exhaust line 329. The control valves 342A, 342B can include any suitable type of valve. The pressure maintenance assembly 326 can be configured to partially and/or fully open and/or close the control valves 342A, 342B. The pressure maintenance assembly 326 can partially and/or fully open and/or close the control valves 342A, 342B via any suitable manner and/or method.

In the embodiment illustrated in FIG. 3, the first control valve 342A is positioned on the fluid injection line 328. The first control valve 342A can be located and/or positioned at any suitable location on the fluid injection line 328. Additionally, the second control valve 342B is positioned on the fluid exhaust line 329. The second control valve 342B can be located and/or positioned at any suitable location on the fluid exhaust line 329. While two control valves 342A, 342B are illustrated in FIG. 3, it is understood that the catheter system 310 and/or the pressure maintenance assembly 326 can include any number of control valves 342A, 342B, i.e., the first control valve, the second control valve, a third control valve, etc. As referred to herein, the control valves 342A, 342B can be used interchangeably and/or may be collectively referred to as “control valve.”

In certain embodiments, the controller 340 can receive and process sensor output to partially and/or fully open and/or close the control valves 342A, 342B. For example, based at least in part on the sensor output, the controller 340 can process and/or determine whether the balloon pressure has varied from the predetermined balloon pressure value and/or is outside the predetermined balloon pressure range. In certain embodiments, when the controller 340 determines that the balloon pressure is below the predetermined balloon pressure value and/or the predetermined balloon pressure range, the controller 340 can partially and/or fully open the control valves 342A, 342B to increase the balloon pressure. More specifically, the controller 340 can partially and/or fully open the first control valve 342A positioned on the fluid injection line 328 to increase the flow rate and the balloon pressure. Alternatively, the controller can partially and/or fully close the second control valve 342B positioned on the fluid exhaust line 329 to decrease the flow rate, which can have the effect of increasing the balloon pressure. In other embodiments, when the controller 340 determines that the balloon pressure is above the predetermined balloon pressure and/or the predetermined balloon pressure range, the controller 340 can partially and/or fully close the control valves 342A, 342B to decrease the balloon pressure. In particular, the controller 340 can partially and/or fully close the first control valve 342A positioned on the fluid injection line 328 to decrease the flow rate and the balloon pressure. Alternatively, the controller 340 can partially and/or fully open the second control valve 342B positioned on the fluid exhaust line 329 to increase the flow rate, which can have the effect of decreasing the balloon pressure. The controller 342 can process the sensor output to partially and/or fully open and/or close the control valves 342A, 342B via any suitable method.

Additionally, in this embodiment, the controller 340 is integrated with and/or included with the handle assembly 320. Further, the pressure sensor 338 is positioned within the fluid injection line 328, but away from and/or outside the inner balloon interior 334.

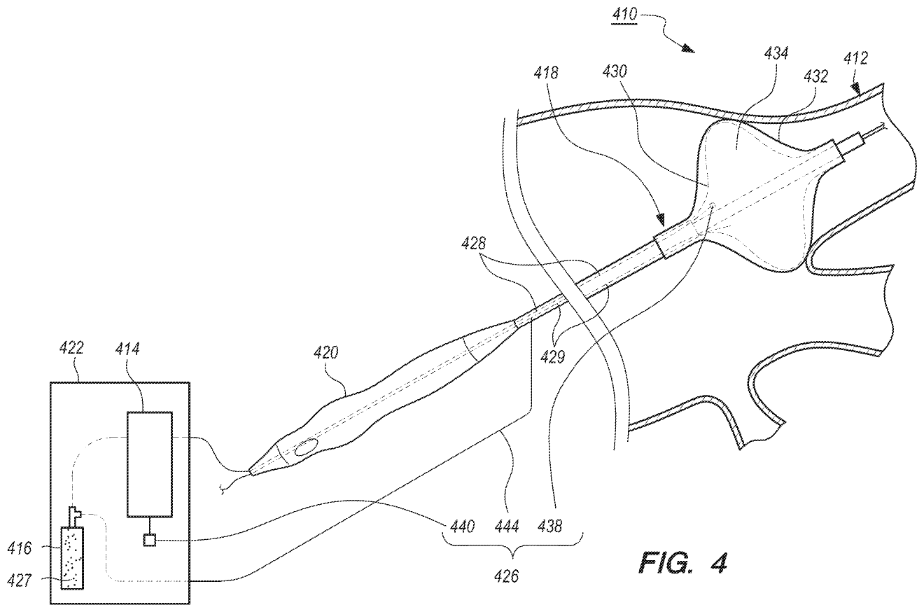

FIG. 4 is a simplified side view of a portion of the patient 412 and yet another embodiment of a portion of the catheter system 410 including yet another embodiment of the pressure maintenance assembly 426. In the embodiment illustrated in FIG. 4, the catheter system 410 includes the control system 414, the fluid source 416, the balloon catheter 418, the handle assembly 420, the control console 422, the pressure maintenance assembly 426, the fluid injection line 428 and the fluid exhaust line 429. However, in this embodiment, the pressure maintenance assembly 426 includes the pressure sensor 438, the controller 440 and an adjunct fluid injection line 444.

In some embodiments, the cryogenic fluid 427 can be delivered to the inner balloon interior 434 via the fluid exhaust line 429 during any stage of the cryoablation procedure, such as during the thawing stage, for example. In the embodiment illustrated in FIG. 4, the adjunct fluid injection line 444 is connected to the fluid exhaust line 429, such that the adjunct fluid injection line 444 and the fluid exhaust line 429 are in fluid communication. It is appreciated that the adjunct fluid injection line 444 and the fluid exhaust line 429 can be connected via any suitable manner or method. Alternatively, the cryogenic fluid 427 can be delivered to the inner balloon interior 434 via the fluid exhaust line 429 during the cryoablation procedure via any other manner or method.

The adjunct fluid injection line 444 can act as a conduit to deliver cryogenic fluid 427 from the fluid source 416 to the fluid exhaust line 429. In other words, the pressure maintenance assembly 426 can also maintain or control a route or path of the cryogenic fluid 427 moving from the fluid source 416 to the inner balloon interior 434. The cryogenic fluid 427 moving through the adjunct fluid injection line 444 can also include a flow rate that varies.

The design of the adjunct fluid injection line 444 can vary. In certain embodiments, the adjunct fluid injection line 444 can include a relatively small diameter tube through which the cryogenic fluid 427 moves. In FIG. 4, the adjunct fluid injection line 444 is shown to extend from the fluid source 416 to a portion of the fluid exhaust line 429. In alternative embodiments, the adjunct fluid injection line 444 can be connected to and/or extend through other structures and/or components of the catheter system 410.

In the embodiment illustrated in FIG. 4, the cryogenic fluid 427 can be selectively delivered from the fluid source 416 through the adjunct fluid injection line 444 and the fluid exhaust line 429 to the inner balloon interior 434 during the cryoablation procedure. In one non-exclusive embodiment, the inner balloon interior 434 can be partially or continuously injected with the cryogenic fluid 427 during the thawing stage. In other words, the inner inflatable balloon 430 can be partially or substantially inflated, i.e., injected with the cryogenic fluid 427, to reduce the likelihood of the balloon catheter 418 from falling out of position, to reduce the likelihood of tissue damage to the patient 412 and/or to reduce the overall cryoablation procedure time.

Additionally, in FIG. 4, the pressure maintenance assembly 426 includes the pressure sensor 438 and the controller 440. In the embodiment illustrated in FIG. 4, the cryogenic fluid 427 is delivered to the inner balloon interior 434 via the adjunct fluid injection line and the fluid exhaust line 429 during the cryoablation procedure, such as during the thawing stage. In this embodiment, the flow rate of the cryogenic fluid 427 moving through the adjunct fluid injection line 444 and the fluid exhaust line 429 can be controlled and/or adjusted by the controller 440.

In the embodiment illustrated in FIG. 4, the pressure sensor 438 is positioned within the inner balloon interior 434. In various embodiments, the controller 440 can receive sensor output and can control or adjust the flow rate of the cryogenic fluid 427 moving through the adjunct fluid injection line 444 and/or the fluid exhaust line 429 based at least partially on the sensor output. For example, based at least in part on the sensor output, the controller 440 can process and/or determine whether the balloon pressure has varied from the predetermined balloon pressure value and/or is outside the predetermined balloon pressure range. In certain embodiments, when the controller 440 determines that the balloon pressure is below the predetermined balloon pressure value and/or the predetermined balloon pressure range, the controller 440 can increase the balloon pressure by increasing the flow rate of the cryogenic fluid 427 moving through the adjunct fluid injection line 444. Alternatively, the controller 440 can increase the balloon pressure by decreasing the flow rate of the cryogenic fluid 427 moving through fluid exhaust line 429. In other embodiments, when the controller 440 determines that the balloon pressure is above the predetermined balloon pressure and/or the predetermined balloon pressure range, the controller 440 can decrease the balloon pressure by decreasing the flow rate of the cryogenic fluid 427 moving through the adjunct fluid injection line 444. Alternatively, the controller 440 can decrease the balloon pressure by increasing the flow rate of the cryogenic fluid 427 moving through fluid exhaust line 429.

Furthermore, in the embodiment illustrated in FIG. 4, the controller 440 is separate from, but in electrical communication with the control system 414.

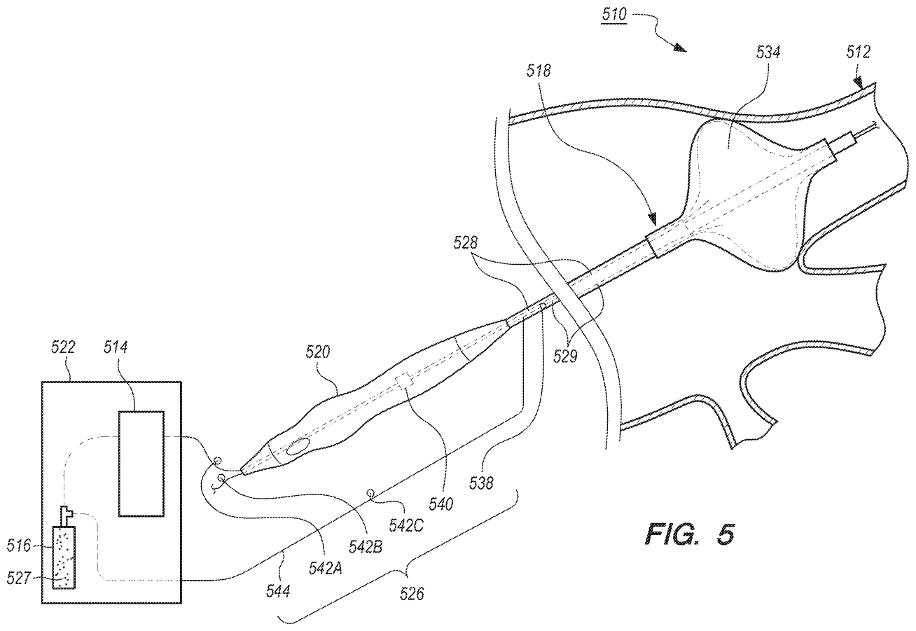

FIG. 5 is a simplified side view of a portion of the patient 512 and even another embodiment of a portion of the catheter system 510 including even another embodiment of the pressure maintenance assembly 526. In the embodiment illustrated in FIG. 5, the catheter system 510 includes the control system 514, the fluid source 516, the balloon catheter 518, the handle assembly 520, the control console 522, the pressure maintenance assembly 526, the fluid injection line 528 and the fluid exhaust line 529. In the embodiment illustrated in FIG. 5, the pressure maintenance assembly 526 includes the pressure sensor 538, the controller 540, one or more control valves 542A, 542B, 542C (i.e., first control valve 542A, second control valve 542B, third control valve 542C) and the adjunct fluid injection line 544.

In FIG. 5, the first control valve 542A is positioned on the fluid injection line 528, the second control valve 542B is positioned on the fluid exhaust line 529 and the third control valve 542C is positioned on the adjunct fluid injection line 544. It is understood, that the control valves 542A, 542B, 542C can be located and/or positioned at any suitable location on the fluid injection line 528, the fluid exhaust line 529 and/or the adjunct fluid injection line 544, respectively. In some embodiments, the controller 540 can receive and process sensor output to partially and/or fully open and/or close the control valves 542A, 542B, 542C. For example, based at least in part on the sensor output, the controller 540 can process and/or determine whether the balloon pressure has varied from the predetermined balloon pressure value and/or is outside the predetermined balloon pressure range. In certain embodiments, when the controller 540 determines that the balloon pressure is below the predetermined balloon pressure value and/or the predetermined balloon pressure range, the controller 540 can partially and/or fully open the control valves 542A, 542B, 542C to increase the balloon pressure. More specifically, the controller 540 can partially and/or fully open the first control valve 542A positioned on the fluid injection line 528 and/or the third control valve 542C positioned on the adjunct fluid injection line 544 to increase the balloon pressure. Alternatively, the controller 540 can partially and/or fully close the second control valve 542B positioned on the fluid exhaust line 529, which can have the effect of increasing the balloon pressure.

In other embodiments, when the controller 540 determines that the balloon pressure is above the predetermined balloon pressure and/or the predetermined balloon pressure range, the controller 540 can partially and/or fully close the control valves 542A, 542B, 542C to decrease the balloon pressure. More particularly, the controller 540 can partially and/or fully close the first control valve 542A positioned on the fluid injection line 528 and/or the third control valve 542C positioned on the adjunct fluid injection line 544 to decrease the balloon pressure. Alternatively, the controller 540 can partially and/or fully open the second control valve 542B positioned on the fluid exhaust line 529, which can have the effect of decreasing the balloon pressure. The controller 540 can process the sensor output to partially and/or fully open and/or close the control valves 542A, 542B, 542C via any suitable method.

Additionally, in this embodiment, the pressure sensor 538 is positioned within the fluid exhaust line 529, but away from or outside the inner balloon interior 534.

It is appreciated that the embodiments of the pressure maintenance assembly described herein enable the realization of one or more certain advantages during the cryoablation procedure, such as during the thawing stage, for example. With the various designs illustrated and described herein, the pressure maintenance assembly can more effectively decrease procedure times by preserving positioning of the balloon catheter and/or maintaining the inflatable balloons at least partially and/or fully inflated during the cryoablation procedure, including during treatment at the plurality of treatment sites. Specifically, the pressure maintenance assembly can more effectively maintain the balloon pressure within the inner inflatable balloon during the thawing stage by maintaining the balloon pressure at approximately the predetermined balloon pressure value or within the predetermined balloon pressure range. Accordingly, the pressure maintenance system can decrease the overall time of the cryoablation procedure by reducing the need to reposition the balloon catheter and/or to fully inflate the inflatable balloons. Further, maintaining the inner inflatable balloon at least partially and/or fully inflated during the thawing stage can also reduce the potential for damage to heart tissue and/or other surrounding tissue of the patient.

It is understood that although a number of different embodiments of the method for controlling the balloon pressure within the inflatable balloon have been illustrated and described herein, one or more features of any one embodiment can be combined with one or more features of one or more of the other embodiments, provided that such combination satisfies the intent of the present invention.

While a number of exemplary aspects and embodiments of the method for controlling the balloon pressure within the inflatable balloon have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. An intravascular cryoablation catheter system comprising:
a cryogenic fluid source;
a balloon catheter including a cryoballoon defining a balloon interior;
a fluid injection line in fluid communication with the cryogenic fluid source and the balloon interior and configured for delivering cryogenic fluid from the cryogenic fluid source to the balloon interior;
a fluid exhaust line in fluid communication with the balloon interior and configured to exhaust the cryogenic fluid from the balloon interior; and
an adjunct fluid injection line in fluid communication with the cryogenic fluid source and the fluid exhaust line and configured to selectively deliver the cryogenic fluid from the cryogenic fluid source to the balloon interior via the fluid exhaust line.

2. The intravascular cryoablation catheter system of claim 1, further comprising a pressure sensor configured to generate a sensor output responsive of a fluid pressure within the balloon interior.

3. The intravascular cryoablation catheter system of claim 2, further comprising a controller configured to selectively control a flow of the cryogenic fluid through at least one of the fluid injection line, the fluid exhaust line or the adjunct fluid injection line based on the sensor output.

4. The intravascular cryoablation catheter system of claim 3, further comprising a first control valve in the fluid injection line operatively coupled to the controller for regulating the flow of the cryogenic fluid through the fluid injection line based on the sensor output.

5. The intravascular cryoablation catheter system of claim 4, further comprising a second control valve in the fluid exhaust line operatively coupled to the controller for regulating the flow of the cryogenic fluid through the fluid exhaust line based on the sensor output.

6. The intravascular cryoablation catheter system of claim 5, further comprising a third control valve in the adjunct fluid injection line for regulating the flow of the cryogenic fluid through the adjunct fluid injection line.

7. The intravascular cryoablation catheter system of claim 6, wherein the pressure sensor is located within the balloon interior.

8. The intravascular cryoablation catheter system of claim 6, wherein the pressure sensor is located away from the balloon interior.

9. The intravascular cryoablation catheter system of claim 6, wherein the cryoballoon includes a first balloon and a second balloon positioned about the first balloon, wherein the first balloon defines the balloon interior.

10. A method for controlling a balloon pressure of an inflatable balloon of an intravascular catheter system, the method comprising the steps of:
sending sensor output to a controller, the sensor output being based at least partially on the balloon pressure; and
maintaining the balloon pressure within a predetermined pressure range based at least partially upon the sensor output received by the controller by adjusting a flow rate of a cryogenic fluid that is selectively delivered from a fluid source to the inflatable balloon through an adjunct fluid injection line that is in fluid communication with a fluid exhaust line.

11. The method of claim 10, further comprising the step of delivering the cryogenic fluid to the inflatable balloon through a fluid injection line.

12. The method of claim 11, further comprising the step of selectively removing the cryogenic fluid from the inflatable balloon through the fluid exhaust line.

13. The method of claim 10, further comprising the step of positioning a pressure sensor within an inner balloon interior of the inflatable balloon.

14. The method of claim 13, wherein the step of positioning includes positioning the pressure sensor within the adjunct fluid injection line.

15. The method of claim 10 wherein the step of maintaining includes controlling with a control valve the flow rate of the cryogenic fluid moving through the adjunct fluid injection line.

16. The method of claim 15, further comprising the step of positioning the control valve on the adjunct fluid injection line.

17. The method of claim 15, wherein the step of controlling includes at least partially opening the control valve with the controller based at least partially upon the sensor output received by the controller.

18. The method of claim 15, wherein the step of controlling includes at least partially closing the control valve with the controller based at least partially upon the sensor output received by the controller.

19. A method for controlling a balloon pressure of an inflatable balloon of an intravascular catheter system, the method comprising the steps of:
sending sensor output to a controller, the sensor output being at least partially based on the balloon pressure; and
maintaining the balloon pressure within a predetermined pressure range based at least partially upon the sensor output received by the controller by adjusting a flow rate of at least one of (i) a cryogenic fluid moving through a fluid injection line, (ii) the cryogenic fluid moving through a fluid exhaust line and (iii) the cryogenic fluid moving through an adjunct fluid injection line that is in fluid communication with the fluid exhaust line.

20. The method of claim 19, wherein the step of maintaining includes controlling with a control valve the flow rate of at least one of (i) the cryogenic fluid moving through the fluid injection line, (ii) the cryogenic fluid moving through a fluid exhaust line and (iii) the cryogenic fluid moving through the adjunct fluid injection line.
